(19) United States
(12) Patent Application Publication
(10) Pub. No.: US 2021/0195981 A1
(43) Pub. Date: Jul. 1, 2021

(54) SYSTEM AND METHOD FOR MONITORING A COGNITIVE STATE OF A RIDER OF A VEHICLE

(71) Applicant: Robert Bosch GmbH, Stuttgart (DE)

(72) Inventors: Shabnam GHAFFARZADEGAN, San Mateo, CA (US); Benzun Pious Wisely BABU, San Jose, CA (US); Zeng DAI, Santa Clara, CA (US); Liu REN, Cupertino, CA (US)

(21) Appl. No.: 16/728,001

(22) Filed: Dec. 27, 2019

(51) Int. Cl. A42B 3/30; G06K 9/00; H04B 1/3827; A61B 5/18; A61B 5/00; A61B 5/11; A61B 5/0476; A61B 5/08; B62J 45/20; B60W 50/08; B60W 50/00; B60W 40/08; G02B 27/01

(52) U.S. Cl. CPC A42B 3/303; G02B 2027/0141; H04B 1/385; A61B 5/18; A61B 5/0006; A61B 5/6803; A61B 5/11; A61B 5/0476; A61B 5/082; A61B 5/4845; A61B 5/742; A61B 5/0022; B62J 45/20; B60W 50/085; B60W 50/0098; B60W 40/08; G02B 27/0172; H04B 2001/3866; B60W 2540/229; B60W 2300/36; B60W 2420/42; B60W 2540/24; B60W 2556/45; G06K 9/00845

(57) ABSTRACT

A helmet includes one or more sensors located in the helmet and configured to obtain cognitive-load data indicating a cognitive load of a rider of a vehicle, a wireless transceiver in communication with the vehicle, a controller in communication with the one or more sensors and the wireless transceiver, wherein the controller is configured to determine a cognitive load of the occupant utilizing at least the cognitive-load data and send a wireless command to the vehicle utilizing the wireless transceiver to execute commands to adjust a driver assistance function when the cognitive load is above a threshold.

### TECHNICAL FIELD

The present disclosure relates to intelligent helmets on saddle-ride vehicles.

### BACKGROUND

Fatigue may make a rider feel tired, weary or sleepy resulting from various everyday conditions, such as insufficient sleep, prolonged mental or physical work, shift work, extended periods of stress or anxiety, etc. Fatigue can impact a rider's concentration and performance level. Fatigue may even cause accidents during vehicle operation, including those in two wheeler riders in which driver's full attention may be crucial at all the times.

There are several devices available on the market for car driver's fatigue monitoring in the form of head-worn and wrist-worn. These devices use motion sensors, EEG, eyelid movement and other sensors to detect the alertness of the driver mostly for car drivers or industry workers. However, there is no device targeting for two-wheeler riders specifically.

### SUMMARY

According to one embodiment, a helmet includes one or more sensors located in the helmet and configured to obtain cognitive-load data indicating a cognitive load of a rider of a vehicle, a wireless transceiver in communication with the vehicle, a controller in communication with the one or more sensors and the wireless transceiver, wherein the controller is configured to determine a cognitive load of the occupant utilizing at least the cognitive-load data and send a wireless command to the vehicle utilizing the wireless transceiver to execute commands to adjust a driver assistance function when the cognitive load is above a threshold.

According to one embodiment, a helmet includes one or more sensors located in the helmet and configured to obtain cognitive-load data indicating a cognitive load of a rider of a saddle-ride vehicle, a wireless transceiver in communication with the vehicle, a controller in communication with the one or more sensors and the wireless transceiver, wherein the controller is configured to determine a cognitive load of the occupant utilizing at least the cognitive-load data, and send a command to the vehicle to execute commands to adjust a driver assistance function when the cognitive load is above a first threshold.

According to one embodiment, a method of monitoring a rider wearing a helmet on a saddle-ride vehicle includes obtaining cognitive-load data indicating a cognitive load of a rider of the saddle-ride vehicle, communicating information with a remote server and the saddle-ride vehicle, determining a cognitive load of the rider utilizing at least the cognitive-load data, and executing commands to be sent to the saddle-ride vehicle to adjust a driver assistance function of the saddle-ride vehicle when the cognitive load is above a threshold.

### BRIEF DESCRIPTION OF THE DRAWINGS

FIG. 1 is an example of a system design 100 that includes a smart helmet 101 and a motorcycle 103.

FIG. 2 is an example of a system that includes a smart helmet that can identify a cognitive load.

FIG. 3 is an exemplary flow chart 300 of a identifying a cognitive load of a rider of a saddle-ride vehicle.

### DETAILED DESCRIPTION

Embodiments of the present disclosure are described herein. It is to be understood, however, that the disclosed embodiments are merely examples and other embodiments can take various and alternative forms. The figures are not necessarily to scale; some features could be exaggerated or minimized to show details of particular components. Therefore, specific structural and functional details disclosed herein are not to be interpreted as limiting, but merely as a representative basis for teaching one skilled in the art to variously employ the embodiments. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

This disclosure makes references to helmets and saddle-ride vehicles. It should be understood that a “saddle-ride vehicle” typically refers to a motorcycle, but can include any type of automotive vehicle in which the driver typically sits on a saddle, and in which helmets are typically worn due to there being no cabin for protection of the riders. Other than a motorcycle, this can also include other powered two-wheeler (PTW) vehicles such as dirt bikes, scooters, and the like. This can also include a powered three-wheeler, or a powered four-wheeler such as an all-terrain vehicle (ATV) and the like. Any references specifically to a motorcycle, vehicle, or bike can also apply to any other saddle-ride vehicle, unless noted otherwise.

The helmet or PTW may also include an electric control unit (ECU). The ECU may more generally be referred to as a controller, and can be any controller capable of receiving information from various sensors, processing the information, and outputting instructions to adjust driving assistance functions, for example. In this disclosure, the terms “controller” and “system” may refer to, be part of, or include processor hardware (shared, dedicated, or group) that executes code and memory hardware (shared, dedicated, or group) that stores code executed by the processor hardware. The code is configured to provide the features of the controller and systems described herein. In one example, the controller may include a processor, memory, and non-volatile storage. The processor may include one or more devices selected from microprocessors, micro-controllers, digital signal processors, microcomputers, central processing units, field programmable gate arrays, programmable logic devices, state machines, logic circuits, analog circuits, digital circuits, or any other devices that manipulate signals (analog or digital) based on computer-executable instructions residing in memory. The memory may include a single memory device or a plurality of memory devices including, but not limited to, random access memory (“RAM”), volatile memory, non-volatile memory, static random-access memory (“SRAM”), dynamic random-access memory (“DRAM”), flash memory, cache memory, or any other device capable of storing information. The non-volatile storage may include one or more persistent data storage devices such as a hard drive, optical drive, tape drive, non-volatile solid-state device, or any other device capable of persistently storing information. The processor may be configured to read into memory and execute computer-executable instructions embodying one or more software programs residing in the non-volatile storage. Programs residing in the non-volatile storage may include or be part of an operating system or an application, and may be compiled or interpreted from computer programs created using a variety of programming languages and/or technologies, including, without limitation, and either alone or in combination, Java, C, C++, C#, Objective C, Fortran, Pascal, Java Script, Python, Perl, and PL/SQL. The computer-executable instructions of the programs may be configured, upon execution by the processor, to cause activation of driver assistance functions when a cognitive threshold is exceeded, for example.

Implementations of the subject matter and the operations described in this specification can be implemented in digital electronic circuitry, or in computer software embodied on a tangible medium, firmware, or hardware, including the structures disclosed in this specification and their structural equivalents, or in combinations of one or more of them. Implementations of the subject matter described in this specification can be implemented as one or more computer programs embodied on a tangible medium, i.e., one or more modules of computer program instructions, encoded on one or more computer storage media for execution by, or to control the operation of, a data processing apparatus. A computer storage medium can be, or be included in, a computer-readable storage device, a computer-readable storage substrate, a random or serial access memory array or device, or a combination of one or more of them. The computer storage medium can also be, or be included in, one or more separate components or media (e.g., multiple CDs, disks, or other storage devices). The computer storage medium may be tangible and non-transitory.

A computer program (also known as a program, software, software application, script, or code) can be written in any form of programming language, including compiled languages, interpreted languages, declarative languages, and procedural languages, and the computer program can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, libraries, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., a field programmable gate array (“FPGA”) or an application specific integrated circuit (“ASIC”). Such a special purpose circuit may be referred to as a computer processor even if it is not a general-purpose processor.

A smart helmet may include a feature to identify and determine a cognitive state to include fatigue. Additionally, the smart helmet may include identifiers for alcohol consumption. The embodiments described below may measure brain waves and other physiological signals, driving patters, time of the day, purpose of the trip, environmental conditions, etc. Sensor may be utilized on the helmet to connect directly to a rider's skull to easily track brain waves and other measurements. Such inputs may be fused to identify the cognitive state of the rider and trigger certain safety features of the vehicle (e.g., motorcycle).

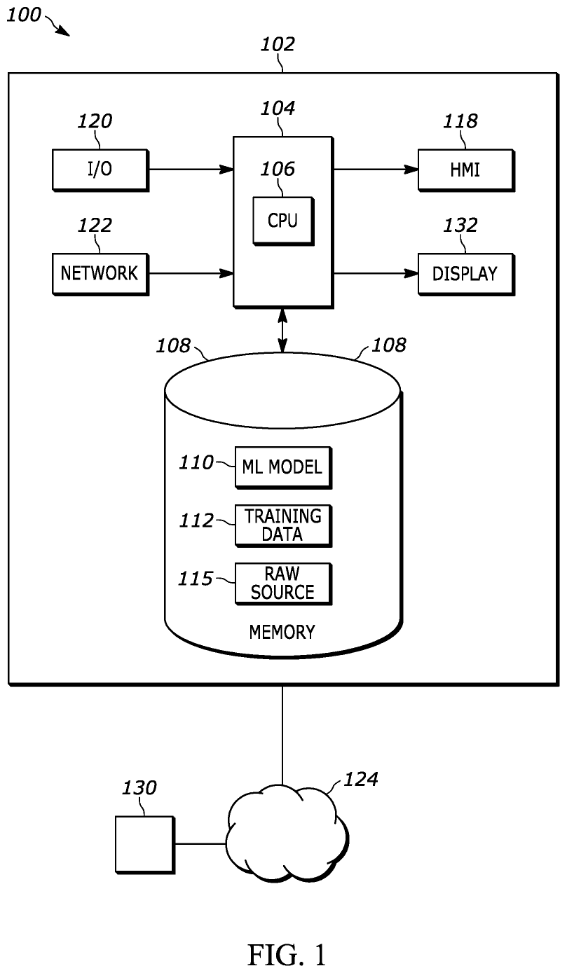

FIG. 1 is an example of a system design 100 that includes a smart helmet 101 and a motorcycle 103. The smart helmet 101 and motorcycle 103 may include various components and sensors that interact with each other. The smart helmet 101 may focus on collecting data related to body and head movement of a driver. In one example, the smart helmet 101 may include a camera 102. The camera 102 of the helmet 101 may include a primary sensor that is utilizing for position and orientation recognition in moving vehicles. Thus, the camera 102 may face outside of the helmet 101 to track other vehicles and objects surrounding a rider. The camera 102 may have difficulty capturing dynamics of such objects and vehicles. In another example, the helmet 101 may be included with radar or LIDAR sensors, in addition to or instead of the camera 102.

The helmet 101 may also include a helmet inertial measurement unit (IMU) 104. The helmet IMU 104 may be utilized to track high dynamic motion of a rider's head. Thus, the helmet IMU 104 may be utilized to track the direction a rider is facing or the rider viewing direction.

Additionally, the helmet IMU 104 may be utilized for tracking sudden movements and other movements that may arise. An IMU may include one or more motion sensors.

An Inertial Measurement Unit (IMU) may measure and report a body's specific force, angular rate, and sometimes the earth's magnetic field, using a combination of accelerometers and gyroscopes, sometimes also magnetometers. IMUs are typically used to maneuver aircraft, including unmanned aerial vehicles (UAVs), among many others, and spacecraft, including satellites and landers. The IMU may be utilized as a component of inertial navigation systems used in various vehicle systems. The data collected from the IMU's sensors may allow a computer to track a motor position.

An IMU may work by detecting the current rate of acceleration using one or more axes, and detect changes in rotational attributes like pitch, roll and yaw using one or more axes. Typical IMU also includes a magnetometer, which may be used to assist calibration against orientation drift by using earth's magnetic field measurements. Inertial navigation systems contain IMUs that have angular and linear accelerometers (for changes in position); some IMUs include a gyroscopic element (for maintaining an absolute angular reference). Angular rate meters measure how a vehicle may be rotating in space. There may be at least one sensor for each of the three axes: pitch (nose up and down), yaw (nose left and right) and roll (clockwise or counter-clockwise from the cockpit). Linear accelerometers may measure non-gravitational accelerations of the vehicle. Since it may move in three axes (up & down, left & right, forward & back), there may be a linear accelerometer for each axis. The three gyroscopes are commonly placed in a similar orthogonal pattern, measuring rotational position in reference to an arbitrarily chosen coordinate system. A computer may continually calculate the vehicle's current position. For each of the six degrees of freedom (x,y,z and Ox, Oy, and Oz), it may integrate over time the sensed acceleration, together with an estimate of gravity, to calculate the current velocity. It may also integrate the velocity to calculate the current position. Some of the measurements provided by an IMU are below:

â_{B}=R_{BW}(a_{w}−g_{w})+bₐ+ηₐ

\hat{ω}_{B}=ω_{B}+b_{g}+η_{g}

(â_{B}, \hat{ω}_{B}) are the raw measurements from the IMU in the body frame of the IMU. a_{w}, ω_{B} are the expected correct acceleration and the gyroscope rate measurements. bₐ, b_{g} are the bias offsets in accelerometer and the gyroscope. ηₐ, η_{g} are the noises in accelerometer and the gyroscope.

The helmet 101 may also include an eye tracker 106. The eye tracker 106 may be utilized to determine a direction of where a rider of the motorcycle 103 is looking. The eye tracker 106 can also be utilized to identify drowsiness and tiredness or a rider of the PTW. The eye tracker 106 may identify various parts of the eye (e.g. retina, cornea, etc.) to determine where a user is glancing. The eye tracker 106 may include a camera or other sensor to aid in tracking eye movement of a rider.

The helmet 101 may also include a helmet processor 108. The helmet processor 107 may be utilized for sensor fusion of data collected by the various camera and sensors of both the motorcycle 103 and helmet 101. In other embodiment, the helmet may include one or more transceivers that are utilized for short-range communication and long-range communication. Short-range communication of the helmet may include communication with the motorcycle 103, or other vehicles and objects nearby. In another embodiment, long-range communication may include communicating to an off-board server, the Internet, “cloud,” cellular communication, etc. The helmet 101 and motorcycle 103 may communicate with each other utilizing wireless protocols implemented by a transceiver located on both the helmet 101 and motorcycle 103. Such protocols may include Bluetooth, Wi-Fi, etc. The helmet 101 may also include a heads-up display (HUD) that is utilized to output graphical images on a visor of the helmet 101.

The motorcycle 103 may include a forward-facing camera 105. The forward-facing camera 105 may be located on a headlamp or other similar area of the motorcycle 103. The forward-facing camera 105 may be utilized to help identify where the PTW is heading. Furthermore, the forward-facing camera 105 may identify various objects or vehicles ahead of the motorcycle 103. The forward-facing camera 105 may thus aid in various safety systems, such as an intelligent cruise control or collision-detection systems.

The motorcycle 103 may include a bike IMU 107. The bike IMU 107 may be attached to a headlight or other similar area of the PTW. The bike IMU 107 may collect inertial data that may be utilized to understand movement of the bike. The bike IMU 107 have multiple axis accelerometer, typically in three orthogonal axes. Similarly, the bike IMU 107 may also include multiple gyroscopes.

The motorcycle 103 may include a rider camera 109. The rider camera 109 may be utilized to keep track of a rider of the motorcycle 103. The rider camera 109 may be mounted in various locations along a handlebar of the motorcycle, or other locations to face the rider. The rider camera 109 may be utilized to capture images or video of the rider that are in turn utilized for various calculations, such as identifying various body parts or movement of the rider. The rider camera 109 may also be utilized to focus on the eye's of the rider. As such, eye gaze movement may be determined to figure out where the rider is looking.

The motorcycle 103 may include an electronic control unit 111. The ECU 111 may be utilized to process data collected by sensors on the motorcycle, as well as data collected by sensors on the helmet. The ECU 111 may utilize the data received from the various IMUs and cameras to process and calculate various positions or to conduct object recognition. The ECU 111 may be in communication with the rider camera 109, as well as the forward-facing camera 105. For example, the data from the IMUs may be fed to the ECU 111 to identify position relative to a reference point, as well as orientation. When image data is combined with such calculations, the bike's movement can be utilized to identify the direction a rider is facing or focusing on. The image data from both the forward-facing camera on the bike and the camera on the helmet are compared to determine the relative orientation between the bike and the riders head. The image comparison can be performed based on sparse features extracted from both the cameras (e.g., rider camera 109 and forward-facing camera 105). The motorcycle 103 may include a bike central processing unit 113 to support the ECU. The system may thus continuously monitor the rider attention, posture, position, orientation, contacts (e.g., grip on handlebars), rider slip (e.g., contact between rider and seat), rider to vehicle relation, and rider to world relation.

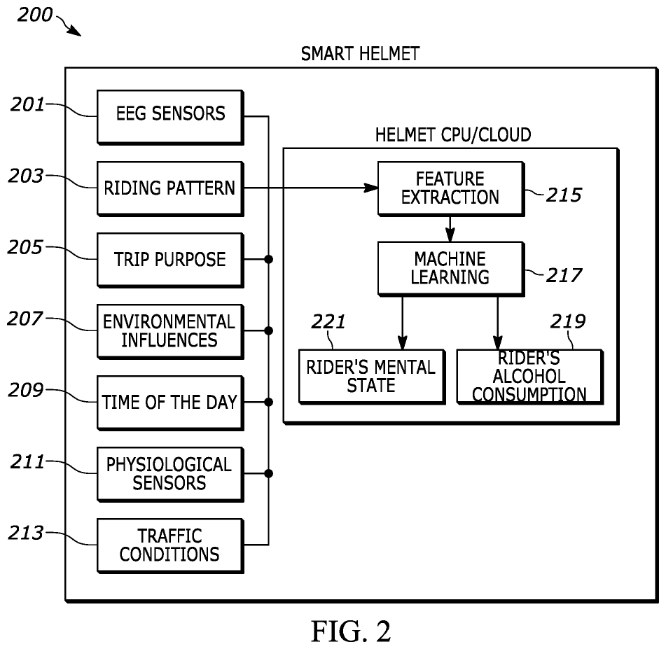

FIG. 2 disclose an example of a smart helmet that includes sensors to help identify a cognitive load of a rider. The smart helmet 200 may include an electroencephalogram (EEG) sensor 201. The smart helmet 200 may include typical features of a helmet that is utilized to provide safety to a rider, including a visor, a hard outer shell, and a soft inner shell that covers the entire head of a rider. The EEG sensors 201 may acquire electroencephalogram (EEG) signals from one or more EEG sensors arranged to acquire EEG signals from the head of a user. The system may utilize the signals to monitor the EEG activity signals exhibited by the user and acquired by the sensor unit. The integrated system may further include a data processing unit to process multiple EEG signals and communicate with the data processing unit of the smart helmet. The processes to analyze the acquired EEG signals may be performed on the data processing unit or utilize the processing unit of the portable electronic device.

The smart helmet 200 may also include a riding pattern sensor 203. The riding pattern sensor 203 may utilized to identify behavior of the rider's driving. For example, if a rider keeps drifting off onto other lanes, it may utilize such information. Rider performance evaluator may assess rider performance based on the PTW's dynamic data, collected either through embedded data source (such as the CAN bus) or installed data source (such as gyroscope, etc). The rider performance evaluator could be used decide whether a driver is sufficiently focused on the driving task or whether the rider is capable of dealing the current driving environment. The data collected from rider performance data may also be used identify a cognitive load of the rider.

The smart helmet 200 may also include a trip purpose 205 identifier. The trip purpose identifier 205 may determine the purpose of the trip to factor into the cognitive load of the user. For example, if the trip purpose identifier 205 recognizes that the commute is a familiar commute on familiar roads that do not require the same cognitive load as a new experience, such as driving in a dense urban area that the rider has never traveled before, it may assume the cognitive load of the rider would be reduced. The trip purpose identifier 205 may work with a navigation system to determine an identified destination.

The smart helmet 200 may also include an environmental influences sensor 207, (e.g. camera, radar, LiDar, in-vehicle camera, speed sensor, windshield wiper sensor, biometric sensor, etc.) as well as off-board servers to understand surrounding conditions as related to the riders environment. The PTW may utilize other sensors, such as fog lights, windshield wipers, rain sensors, moisture sensors, etc. that may also be utilized as inputs to determining the cognitive load. For example, when a fog light is activated, or the windshield wipers are moving faster, or a rain sensor identifies higher precipitation, the rider's cognitive load may be high. Off-board data may be utilized to identify factors that may keep a user pre-occupied with riding and increase the cognitive load. For example, weather data from an off-board server can identify weather conditions. The weather data may identify sever weather updates, bad driving conditions (e.g. icy road), and other items that may affect a driver's cognitive load.

The smart helmet 200 may also utilize sensors to identify the time of day 209 to factor in the cognitive load. Such sensors may include a clock or a photocell sensor, photoresistor, light detecting resistor, or other sensor that is able to detect light or the absence of light. Thus, the sensor may understand that it is day time or nighttime based on a time or a light intensity outside of the motorcycle. The sensor may be located on an outer surface of the helmet. In another embodiment, a GPS receiver may be utilized to identify dusk and dawn time for the PTW.

The smart helmet 200 may also include physiological sensors 211. The physiological sensors may include sensors that are able to identify hear rate, respiration rates, or blood pressure. The physiological sensors 211 may also include blood volume pressure, head blood volume pulse, electrocardiography, electrodermal activity (with a Q-sensor), electromyography (EMG), emotion sensor (ECG), etc. Such psychological sensors may include a heat flux sensor to measure a temperature of the user or various sensors including on-chip image and color sensors, and sensors that measure pH, temperature, and pressure may offer a quick and accurate diagnostic tool to detect gastrointestinal abnormalities. Data-processing steps like filtering, noise cancellation, and amplification may be applied to improve accuracy.

The smart helmet 200 may also utilize various sensors to monitor the traffic conditions surrounding the PTW. Off-board data may be utilized to identify traffic factors that may keep a user pre-occupied with riding and increase the cognitive load. For example, traffic data from an off-board server can identify sever traffic conditions or accidents. The traffic data may identify traffic flow updates, accidents, and other events that may affect traffic flow and in turn a driver's cognitive load.

An OMS may be mounted in the helmet or suitable location could observe user's interaction with the PTW or any other distractions. The OMS evaluates the actual cognitive demands or potential cognitive demands from interacting with the PTW. For example, if the OMS detecting the user is actively driving fast while taking a phone call on the handsfree system, his/her cognitive load may be evaluated as high. In another example, if the OMS detects another occupant on the PTW other than the rider, the OMS may predict the cognitive demand of the user may increase soon.

The smart helmet 200 or a remote server may be utilized to gather the various sensor data to identify a cognitive load or mental state by utilizing a feature extraction 215. The feature extraction 215 may take raw input signals or some statistics of the signals and utilize them to train a machine learning model 217 to predict a rider's mental state 221 or alcoholic consumption 219. Different machine learning classifiers from the traditional ones such as decision tree and support vector machine to more advanced methods such as deep learning can be used in 217. Multi-task classifier is trained on the extracted features to predict 219 and 221. The predictions of the rider's mental state may be used to alert the rider by planning sounds or vibrations on the bike handle. The alcohol influence results can be used to prevent the rider from operating the vehicle. For example, the smart helmet may identify that the rider is under the influence utilizing a breathalyzer that is built into the helmet, and in turn, the helmet sends commands to the PTW to activate features or to disable operation of the PTW.

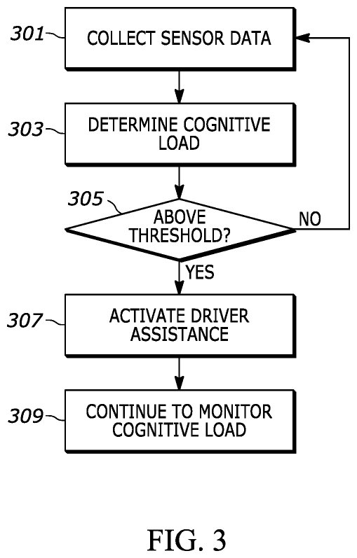

FIG. 3 is an exemplary flow chart 300 of a identifying a cognitive load of a rider of a PTW. The flow chart 300 may be implemented on a PTW-side application in a vehicle controller or off-board at a remote server. The system may collect sensor data and any other data utilized to identify the cognitive load of the rider at step 201. The system may communicate with sensors in the helmet or PTW, as well as off-board servers. Such sensors and data may include those described in FIG. 1 and FIG. 2 above.

The system may also determine if a cognitive load of the user at step 203. The system may utilize various sensors in the helmet to help identify a cognitive load of the rider controlling the PTW. The helmet may also communicate with the PTW to gather other information to identify a cognitive load. For example, the system may utilize a vehicle speed sensor to identify how fast the vehicle is traveling. At a high-level, the faster the vehicle is traveling, the cognitive load of the driver can be assumed to be greater (e.g. the driver is focusing on driving rather than the task). Thus, the higher the cognitive load, the more distracted the user may be with additional tasks that will prevent the user from being able to focus on additional information on an interface when a video conference session is taking place. The embodiments described above may also be applied to multi-level presentation HMI based on the user's cognitive workload. For example, the highest level of HMI could include all features of conference call. The rest of levels will only include reduced set of the conference call features.

At another level, the cognitive load of the driver may be determined by an DSM (e.g. driver-facing camera) located on the PTW that monitors the occupant's behaviors, including facial movement, eye-movement, etc. The cognitive load of the driver may be determined by a DSM that monitors the surrounding vehicle environment, including the traffic conditions, proximity to other vehicles, complexity level of the road structure, and number of objects surrounding the PTW etc. For example, if many vehicle or objects are surrounding the vehicle, the cognitive load of the driver may be higher. If the DSM fails to identify objects or just a limited amount of objects, the cognitive load of the rider may be low.

Furthermore, information may be utilized to identify a rider of the vehicle to adjust a threshold for a cognitive load of the driver. For example, an age or driving experience of the rider may factor into a rider's cognitive load threshold being lower. Identification of a rider may be determined by user profile data or information obtained from a mobile phone, camera (e.g. facial recognition), or vehicle settings. The system may determine how long the rider has been riding a PTW (e.g., experience) as well as age, accident history, traffic tickets, etc. The system may have a threshold cognitive load that is set to determine whether or not to operate the PTW, alert the driver, or activate some rider assistance functions. For example, if a cognitive load of a rider is determined to be high, an adaptive cruise control feature may be activated. The system may utilize the cognitive load data to identify or estimate a cognitive load of the rider.

At step 305, the system may determine if the cognitive load of the rider exceeds a threshold. The system may adjust the threshold based on various factors in the PTW, such as a rider of the PTW. The interface or driver assisted functions may also allow for automatic adjustment of the threshold that may be set by the rider or via the interface. As such, the cognitive load data may be collected and analyzed to measure and compare against the threshold to determine how the PTW can make adjustment to alert the rider (e.g., play sounds or vibration on the motorcycle handle) or activate a driving assistance feature, or in some circumstances, stop operation of the motorcycle. The system may have more than one threshold. Thus, if multiple thresholds are used, the system may utilize multiple interfaces that have varying level of content for each threshold. Thus, rather than having only two different counteractions to the cognitive load, the system may have three, four, five, six, etc different counteractions that are adjusted by varying thresholds or levels.

At step 307, the vehicle system may execute commands to adjust a driving assistance feature when the cognitive load is above a threshold amount. Thus, if a rider is determined to be overworked (e.g. the rider is not in motion, clear path driving, autonomous/semi-autonomous driving system is helping out), the system may presume the rider may needs assistance to operate the PTW and provide a notification to the rider or activate a driver assistance function. The notification may include vibrating of the saddle or the handlebars of the PTW, as well as a notification that is shown on the display of the HUD of the helmet or audibly output on speakers in the helmet. The driver assistance function may include activating (via a wireless command sent to the PTW from the helmet) an adaptive cruise system, semi-autonomous driving, lane keep assist function, etc. In one example, if the cognitive load is high, the helmet may send a wireless command to the PTW to safely cease operation of the PTW. The system may include various thresholds to operate various functions. Thus, the system may include a variety of features to be activated at a first threshold, and other features at higher thresholds. If a cognitive load is deemed to high or the driver is presumed to be under the influence based on the data, the operation of the vehicle may shut down. The system may send a notification prior to activating the driver assistance function, which may allow the rider to abort activation of the feature or confirm activation of the feature.

At step 309, the vehicle system may continue to monitor the cognitive load even after activation of the notification or the driver assistance function. The notification may include vibrating of the saddle or the handlebars of the PTW, as well as a notification that is shown on the display of the HUD of the helmet or audibly output on speakers in the helmet. The system may deactivate a function or allow operation of certain vehicle functions if the cognitive load eventually falls below the threshold. Thus, if a driver is determined to not be overworked (e.g. the PTW is not in motion, clear path driving, autonomous/semi-autonomous driving system is helping out, etc.), the system may presume the rider can operate the PTW and does not need any assistance. Thus, the system may simply just monitor the data continuously.

The processes, methods, or algorithms disclosed herein can be deliverable to/implemented by a processing device, controller, or computer, which can include any existing programmable electronic control unit or dedicated electronic control unit. Similarly, the processes, methods, or algorithms can be stored as data and instructions executable by a controller or computer in many forms including, but not limited to, information permanently stored on non-writable storage media such as ROM devices and information alterably stored on writeable storage media such as floppy disks, magnetic tapes, CDs, RAM devices, and other magnetic and optical media. The processes, methods, or algorithms can also be implemented in a software executable object. Alternatively, the processes, methods, or algorithms can be embodied in whole or in part using suitable hardware components, such as Application Specific Integrated Circuits (ASICs), Field-Programmable Gate Arrays (FPGAs), state machines, controllers or other hardware components or devices, or a combination of hardware, software and firmware components.

While exemplary embodiments are described above, it is not intended that these embodiments describe all possible forms encompassed by the claims. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to cost, strength, durability, life cycle cost, marketability, appearance, packaging, size, serviceability, weight, manufacturability, ease of assembly, etc. As such, to the extent any embodiments are described as less desirable than other embodiments or prior art implementations with respect to one or more characteristics, these embodiments are not outside the scope of the disclosure and can be desirable for particular applications.

What is claimed is:

1. A helmet, comprising:
one or more sensors located in the helmet and configured to obtain cognitive-load data indicating a cognitive load of a rider of a saddle-ride vehicle;
a wireless transceiver in communication with the vehicle;
a controller in communication with the one or more sensors and the wireless transceiver, wherein the controller is configured to:
determine a cognitive load of the rider utilizing at least the cognitive-load data; and
send a wireless command to the vehicle utilizing the wireless transceiver to execute commands to adjust a driver assistance function when the cognitive load is above a threshold.

2. The helmet of claim 1, wherein the controller is further configured to stop operation of the vehicle when the cognitive load is above the threshold.

3. The helmet of claim 1, wherein the threshold is adjustable based on user profile.

4. The helmet of claim 1, wherein the controller is further configured to obtain user profile information from a mobile phone associated with the rider via the wireless transceiver and adjust the threshold in response to the user profile information.

5. The helmet of claim 1, wherein the wireless transceiver is configured to communicate with an onboard vehicle camera that is configured to monitor movement of the rider and the controller is further configured to utilize information associated with the movement of the occupant to determine the cognitive load.

6. The helmet of claim 1, wherein the controller is further configured to obtain user profile information from a key-fob associated with the rider of the vehicle and adjust the threshold in response to the user profile information.

7. The helmet of claim 1, wherein the helmet includes a helmet display that includes a heads-up display (HUD) configured to display a notification regarding activation of the driver assistance function.

8. The helmet of claim 1, wherein the one or more sensors located in the helmet includes an EEG sensor.

9. A helmet, comprising:
one or more sensors located in the helmet and configured to obtain cognitive-load data indicating a cognitive load of a rider of a saddle-ride vehicle;
a wireless transceiver in communication with the vehicle;
a controller in communication with the one or more sensors and the wireless transceiver, wherein the controller is configured to:
determine a cognitive load of the rider utilizing at least the cognitive-load data; and
send a command to the vehicle to execute commands to adjust a driver assistance function when the cognitive load is above a first threshold.

10. The helmet of claim 9, wherein the controller is further configured to send a command to the vehicle to stop operation of the vehicle when the cognitive load is above a second threshold.

11. The helmet of claim 10, wherein the second threshold indicates a higher cognitive load than the first threshold.

12. The helmet of claim 9, wherein the cognitive load data include alcoholic consumption data obtained from a breathalyzer in the helmet.

13. The helmet of claim 9, wherein the helmet includes a helmet display that includes a heads-up display (HUD) configured to display a notification regarding activation of the driver assistance function.

14. The helmet of claim 9, wherein the wireless transceiver is in communication with a remote server configured to determine the cognitive load and send it to the helmet via the wireless transceiver.

15. The helmet of claim 9, wherein the controller is further configured to adjust the first threshold in response to a user profile received from a mobile device associated with the rider and in communication with the vehicle.

16. A method of monitoring a rider wearing a helmet on a saddle-ride vehicle, comprising:
obtaining cognitive-load data indicating a cognitive load of a rider of the saddle-ride vehicle;
communicating information with a remote server and the saddle-ride vehicle;
determining a cognitive load of the rider utilizing at least the cognitive-load data; and
executing commands to be sent to the saddle-ride vehicle to adjust a driver assistance function of the saddle-ride vehicle when the cognitive load is above a threshold.

17. The method of claim 16, wherein the method includes the step of notifying the rider of adjustment of the driver assistance function.

18. The method of claim 16, wherein the method includes the step of adjusting the threshold based on a user profile.

19. The method of claim 16, wherein the method includes the step of adjusting the threshold based on a user profile.

20. The method of claim 16, wherein the method includes the step of notifying the rider of adjustment of the driver assistance function via a notification on a display of the helmet, wherein the notification includes a confirmation option to confirm the adjustment and a cancellation option to abort the adjustment of the driver assistance function.
